(19) United States
(12) Patent Application Publication
(10) Pub. No.: US 2023/0192653 A1
(43) Pub. Date: Jun. 22, 2023

(54) Novel Therapeutic Methods

(71) Applicant: GUANGZHOU MAXINOVEL PHARMACEUTICALS CO., LTD., Guangdong (CN)

(72) Inventors: Yuguang WANG, Guangdong (CN); Zhenhua FENG, Guangdong (CN); Feilan WANG, Guangdong (CN)

(21) Appl. No.: 17/911,053

(22) PCT Filed: Mar. 8, 2021
(86) PCT No.: PCT/CN2021/079532

§ 371 Date: Sep. 12, 2022

(30) Foreign Application Priority Data
Mar. 13, 2020 (WO) PCT/CN2020/079196
Jan. 13, 2021 (WO) PCT/CN2021/071588

(51) Int. Cl. C07D 401/14; C07K 16/28; A61P 35/00

(52) U.S. Cl. CPC C07D 401/14; C07K 16/2896; A61P 35/00; C07B 2200/13

(57) ABSTRACT

The disclosure provides new therapeutic uses for certain fused ring pyrimidine compounds, in particular for treating patients having a cancer which expresses elevated fibroblast growth factor receptor oncogene partner 2 (FGFR1OP2) and/or elevated FGFR1, or expresses a FGFR1-FGFR1OP2 fusion protein, and for treating patients who are being treated with an immune checkpoint inhibitor.

The present invention claims the priority of the PCT/CN2020/079196 filed on Mar. 13, 2020, and priority of the PCT/CN2021/071588 filed on Jan. 13, 2021, the contents of which are incorporated herein by their entirety.

### FIELD OF INVENTION

This disclosure relates to new therapeutic uses for certain fused ring pyrimidine compounds, in particular for treating patients having a cancer which expresses elevated levels of fibroblast growth factor receptor oncogene partner 2 (FGFR1OP2), or a FGFR1-FGFR1OP2 fusion protein.

### PRIOR ARTS

Fibroblast growth factor receptor oncogene partner 2 (FGFR1OP2) is a natural protein having a poorly understood function. It is believed to be involved in wound healing. When fused with fibroblast growth factor receptor 1 (FGFR1), it may result in constitutive kinase activity, leading to 8p11myeloproliferative syndrome. Grand, E. K., et al., “Identification of a novel gene, FGFR1OP2, fused to FGFR1 in 8p11 myeloproliferative syndrome.” Genes Chromosomes Cancer (2004) 40:78-83.

Fused ring pyrimidine compounds as described in U.S. Pat. No. 10,494,378B2 (the contents of which are incorporated herein by reference) are known as inhibitors of Janus kinase (JAK), FGFR kinase, FLT3 kinase and Src family kinase, and have utility in treating various immune system diseases, autoimmune diseases, cell proliferative diseases, allergic disorders and cardiovascular diseases. For example, one such compound, MAX-40279, is in clinical trials as a dual inhibitor of FLT3 kinase and FGFR kinase to treat acute myelogenous leukemia (AML) in patients having a mutation resulting increased FLT3 kinase expression and/or activation.

There is a need for new treatments and alternatives to treatment in patients having treatment-resistant cancers.

PD-1 (Programmed death 1, CD279) is a major immunosuppressive molecule. It is a member of the CD28 superfamily and was originally cloned from the apoptotic mouse T cell hybridoma 2B4.11. PD-1 is mainly distributed in immune-related cells, such as T cells, B cells and NK cells, and plays an important role in immune response processes, e.g., autoimmune diseases, tumors, infections, organ transplantation or allergies.

Programmed death-ligand 1 (PD-L1), also known as B7-H1, belongs to the B7 family and is widely distributed in peripheral tissues and hematopoietic cells. PD-L1 is mainly expressed in hematopoietic cells such as CD4 T cells, CD8 T cells, B cells, monocytes, dendritic cells (DCs), macrophages, and some non-hematopoietic cells, such as endothelial cells, islet cells and mast cells. PD-L1 is highly expressed in various tumors, such as lung cancer, gastric cancer, melanoma and breast cancer. Programmed death-1 (PD-1) is the major receptor for PD-L1.

PD-1/PD-L1 exerts a negative immunomodulatory effect. When PD-1 on the surface of immune cells interacts with PD-L1 on the surface of cancer cells, for example, tumor cells, the interaction causes a series of signaling responses leading to inhibition of T lymphocyte proliferation and secretion of related cytokines, apoptosis of tumor antigen-specific T cells, and/or incapable immunization, ultimately suppressing the immune response and promoting the escape of tumor cells. Monoclonal antibodies targeting PD-1 or PD-L1 can break the immune tolerance of tumors by specifically blocking the interaction of PD-1/PD-L1, restore the killing function of tumor-specific T cells on tumor cells, and achieve clearance of tumors. Up to now, there are four PD-1 antibody drugs and four PD-L1 antibody drugs in China and in the US. The approved PD-1 antibody drugs include Merck's Keytruda® (referred to as K drug), Bristol-Myers Squibb's Opdivo® (referred to as O drug), Junshi Bioscience's Toripalimab and Innovent's Sintilimab. The approved PD-L1 antibody drugs include Atezolizumab® by Roche, Durvalumab® by AstraZeneca, Avelumab® by Pfizer and Merck (Germany), and Cemiplimab® by Regeneron. In addition, a number of other companies are developing PD-1/PD-L1 targeted antibody drugs.

Many cancer patients benefit from monoclonal antibodies to PD-1/PD-L1. However, studies have found that anti-PD-1/PD-L1 antibodies are not effective in all cancer patients. Clinical trial data show the effective response rate of anti-PD-1/PD-L1 antibody alone is about 20%.

Small molecule inhibitors binding to PD-1/PD-L1 are also actively developed. WO2018006795, WO2019128918, CN202010939415.0 and CN202011414403.2, which are incorporated herein by reference in their entirety, disclose novel small molecule inhibitors targeting the interaction of PD-1 and PD-L1. The small molecule inhibitors disclosed therein exhibit an anti-tumor effect in a mouse tumor model.

There is a need to improve the effective response rate in cancer immunotherapy, particularly in the case of patients who do not respond to monoclonal antibodies to PD-1/PD-L1.

### Content of the Present Unvention

It is surprisingly found that the association between FGFR1OP2 and FGFR1 may be not only by fusion, wherein FGFR1OP2 is fused to FGFR1 as a result of a translocation event to form a fusion polypeptide exhibiting constitutive kinase activity, as in 8p11 myeloproliferative syndrome, but may also result from non-covalent binding, wherein FGFR1OP2 binds FGFR1, and the binding complex also exhibits constitutive kinase activity, for example in situations where the FGFR10P2 is over-expressed. It is found that FGFR10P2 is highly expressed in a variety of cancers, not only myoproliferative syndrome.

It is further discovered that the fused ring pyrimidine compounds as described herein are effective to specifically inhibit fibroblast growth factor receptor oncogene partner 2 (FGFR1OP2), when bound to or fused with fibroblast growth factor receptor 1 (FGFR1). Moreover, it is found that FGFR10P2 is highly expressed in a variety of cancers, not only myoproliferative syndrome, which are responsive to treatment with the fused ring pyrimidine compounds as described herein. Fused ring pyrimidine compounds as described herein are believed to bind to and block the FGFR1-FGFR1OP2 complex, both when FGFR1OP2 is bound to FGFR1 and when FGFR1OP2 is fused with FGFR1, and simultaneously to bind with the TK2 of FGFR1 to exert therapeutic efficacy.

It is moreover surprisingly found that fused ring pyrimidine compounds as described herein enhance the effects of immune checkpoint inhibitors, e.g. inhibitors of PD-1 and/or PD-L1, for example antibodies to PD-1 or PD-L1, or small molecule inhibitors targeting the interaction of PD-1 and PD-L1.

Accordingly, the disclosure provides a method of treating a cancer in a patient in need thereof, wherein the cancer expresses elevated levels of FGFR10P2 and/or FGFR1, comprising administering to said patient an effective amount of a fused ring pyrimidine compounds as described herein, e.g., a Compound of Formula (I) or compound 1, as hereinafter described, e.g., as described in U.S. Pat. No. 10,494,378B2, the contents of which are incorporated herein by reference, in free or pharmaceutically acceptable salt form.

The disclosure further provides fused ring pyrimidine compounds as described herein, in free or pharmaceutically acceptable salt form, for use in treating cancers which express elevated levels of FGFR1OP2 and/or FGFR1, and the use of fused ring pyrimidine compounds as described herein, in free or pharmaceutically acceptable salt form, in the manufacture of a medicament for treating cancers which express elevated levels of FGFR1OP2 and/or FGFR1.

The disclosure further provides a method of treating a cancer in a patient in need thereof (e.g., wherein the cancer expresses elevated levels of FGFR1OP2 and/or FGFR1), comprising administering to said patient (i) an effective amount of a fused ring pyrimidine compounds as described herein, e.g., a Compound of Formula (I) or compound 1, as hereinafter described, e.g., as described in U.S. Pat. No. 10,494,378B2, the contents of which are incorporated herein by reference, in free or pharmaceutically acceptable salt form, and (ii) an effective amount of an immune checkpoint inhibitor, e.g. an inhibitor of PD-1 or PD-L1, for example antibodies to PD-1 or PD-L1, or small molecule inhibitors targeting the interaction of PD-1 and PD-L1.

Further areas of applicability of the present disclosure will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

In a first embodiment, the disclosure provides a method (Method 1) of treating a cancer in a patient in need thereof, wherein the cancer (i) exhibits elevated levels of FGFR1OP2 and/or FGFR1; and/or (ii) is characterized by a translocation mutation expressing a FGFR1OP2-FGFR1 fusion protein, comprising administering an effective amount of a Compound of Formula (I), in free or pharmaceutically acceptable salt form:

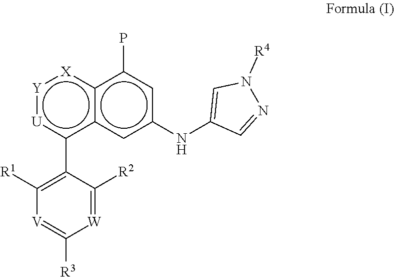

wherein, P is selected from a hydrogen or a deuterium;

X is selected from CH or S;

Y is selected from N or CR⁵;

U is selected from a chemical bond or CH;

V is selected from N or CH;

W is selected from N or CR⁶;

each of R¹, R², R³ and R⁶ is independently selected from the group consisting of a hydrogen, a deuterium, a halogen, a substituted or unsubstituted alkyl,

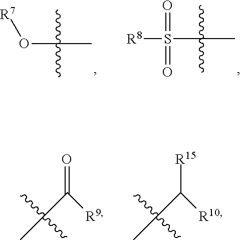

a cycloalkyl and a heterocycloalkyl; each of R⁷, R⁸, R⁹, R¹⁰ and R¹⁵ is independently selected from the group consisting of a hydrogen, a deuterium, a halogen, a hydroxyl, an amino, a substituted or unsubstituted alkyl, an alkoxy,

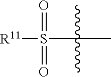

and a heterocycloalkyl; R¹¹ is a hydrogen, a deuterium or an alkyl; or R⁶, R² and the two atoms on the ring to which they are attached form a “substituted or unsubstituted 5- to 7-membered carbon heterocycle”; or, R⁶, R³ and the two atoms on the ring to which they are attached form a “substituted or unsubstituted 5- to 7-membered carbon heterocycle”; the heteroatom in “substituted or unsubstituted 5- to 7-membered carbon heterocycle” is selected from the group consisting of nitrogen, oxygen and sulfur;

R⁴ is a hydrogen, a deuterium, a substituted or unsubstituted alkyl, an alkoxy, a cycloalkyl, or a substituted or unsubstituted heterocycloalkyl;

R⁵ is a hydrogen, a deuterium, a halogen, or an alkyl;

in the definitions of R¹, R², R³ and R⁶, the “substituted” in “a substituted or unsubstituted alkyl” means to be substituted with the substituents selected from the group consisting of a halogen, a hydroxyl, an amino, an alkyl, an alkoxy,

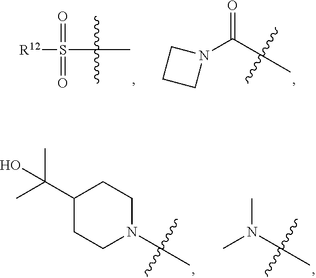

and a heterocycloalkyl, in the case when multiple substituents are present, the substituents are the same or different; R¹² is a hydrogen, a deuterium, or an alkyl;

in the definitions of R⁷, R⁸, R⁹, R¹⁰ and R¹⁵, the “substituted” in “a substituted or unsubstituted alkyl” means to be substituted with the substituents selected from the group consisting of a deuterium, a halogen, a hydroxyl, an amino, an alkyl, an alkoxy,

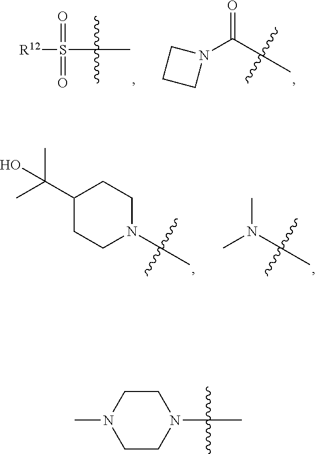

and a heterocycloalkyl, in the case when multiple substituents are present, the substituents are the same or different; R¹³ is a hydrogen or an alkyl;

in the definition of R⁴, the “substituted” in “a substituted or unsubstituted alkyl” and “a substituted or unsubstituted heterocycloalkyl” means to be substituted with the substituents selected from the group consisting of a hydroxyl, an alkyl,

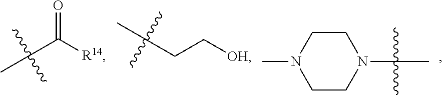

and heterocycloalkyl, in the case when multiple substituents are present, the substituents are the same or different; R¹⁴ is a hydrogen, an alkyl, a hydroxymethyl or an alkoxy;

the “substituted” in “substituted or unsubstituted 5- to 7-membered carbon heterocycle” means to be substituted with one or more than one alkyl

[e.g., as described in US10494378B2, the contents of which are incorporated herein by reference] in free or pharmaceutically acceptable salt form.

For example, the disclosure provides

1.1Method 1 wherein the Compound of Formula (I) is Compound 1 in free or pharmaceutically acceptable salt form:

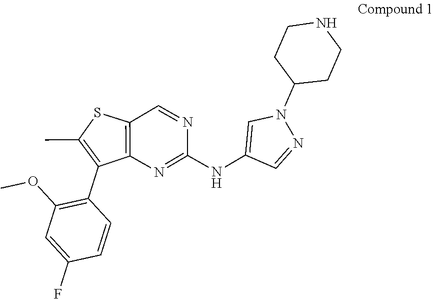 [SMILES: COc1cc(F)ccc1-c1c(C)sc2cnc(Nc3cnn(C4CCNCC4)c3)nc12]

1.2Method 1.1 wherein the Compound 1 is in pharmaceutically acceptable acid addition salt form.

1.3Method 1.2 wherein the pharmaceutically acceptable acid addition salt form of Compound 1 is selected from the fumarate, phosphate, tartrate, and adipate salts.

1.4Method 1.3 wherein the pharmaceutically acceptable acid addition salt form of Compound 1 is in crystalline form [e.g., as disclosed in WO2019228171A1, the contents of which are incorporated herein by reference].

1.5Method 1.3 or 1.4 wherein the acceptable acid addition salt form of Compound 1 is the hemifumarate.

1.6Any foregoing method wherein the cancer exhibits elevated levels of FGFR1OP2 as measured using gene expression profiling, e.g., using RNA sequencing or real-time quantitative PCR (RT-qPCR).

1.7Any foregoing method wherein the cancer exhibits elevated levels of FGFR1OP2 as measured using an immunoassay for FGFR1OP2, e.g., using Western blotting, ELISA or in situ hybridization.

1.8Any foregoing method wherein the cancer exhibits elevated levels of FGFR1OP2 as measured using gene expression profiling, and the gene expression of FGFR1OP2 in the cancer is greater than five mRNA transcripts per million mRNA transcripts (TPM), e.g., at least 9 TPM, e.g., at least 10 TPM, e.g., at least 12 TPM., e.g., wherein TPM is calculated as described in Wagner GP, et al., “Measurement of mRNA abundance using RNA-seq data: RPKM measure is inconsistent among samples.” Theory Biosci. 2012 December; 131(4):281-5 and Abrams Z B, Johnson T S, Huang K, Payne P R O, Coombes K. “A protocol to evaluate RNA sequencing normalization methods. BMC Bioinformatics”. 2019;20(Suppl 24):679. Published 2019 Dec. 20. doi:10.1186/s12859-019-3247-x.

1.9Any foregoing method wherein the cancer expresses a FGFR1OP2- FGFR1 fusion protein; e.g. wherein the FGFR1OP2-FGFR1 fusion protein exhibits constitutive kinase activity, dimerization induction, constitutive signal transduction, and/or transforming activity; e.g., a protein wherein the first 2 coiled-coil domains of FGFR1OP2 are fused to the carboxy terminal part of FGFR1, including its tyrosine kinase domain, e.g., comprising 132 amino acids from FGFR1OP2 and 394 amino acids from FGFR1.

1.10 Any foregoing method wherein the cancer expresses a FGFR1OP2-FGFR1 fusion protein which exhibits constitutive kinase activity, e.g., wherein the fusion results from a chromosomal translocation to form a gene encoding the FGFR1OP2-FGFRlfusion protein.

1.11 Any foregoing method wherein the cancer expresses a FGFR1OP2- FGFR1 fusion protein as detected using an immunoassay for FGFR1OP2- FGFR1 fusion protein, e.g., using Western blotting, in situ hybridization, or ELISA.

1.12 Any foregoing method wherein the cancer expresses a FGFR1OP2- FGFR1 fusion protein as detected using gene expression profiling, e.g., using RNA sequencing or real-time quantitative PCR (RT-qPCR).

1.13 Any foregoing method wherein the cancer expresses a FGFR1OP2- FGFR1 fusion protein as detected using PCR or DNA probes to detect a mutation resulting in a gene encoding a FGFR1OP2-FGFR1 fusion protein.

1.14 Any foregoing method comprising the steps of

a) obtaining a biological sample, selected from blood or tumor tissue, from the patient, wherein the biological sample is believed to contain cancer cells;

b) either (i) detecting elevated levels of FGFR1OP2 expression in the biological sample using gene expression profiling and/or an immunoassay; or (ii) detecting FGFR1OP2- FGFR1 fusion protein or a gene encoding FGFR1OP2- FGFR1 fusion protein in the biological sample using gene expression profiling, PCR, DNA probe, or immunoassay; and

c) administering an effective dose of a Compound of Formula (I), e.g., of Compound 1, in free or pharmaceutically acceptable salt form to the patient if the biological sample exhibits either elevated levels of FGFR1OP2 expression or the presence of FGFR1OP2- FGFR1 fusion protein or a gene encoding FGFR1OP2- FGFR1 fusion protein.

1.15 Any foregoing method wherein the cancer exhibits FGFR1OP2 binding FGFR1 to exhibit constitutive kinase activity

1.16 Any foregoing method wherein the cancer is selected from carcinoma, sarcoma, melanoma, lymphoma, leukemia and myeloma (e.g., leukemia).

1.17 Any foregoing method wherein the cancer is a solid tumor.

1.18 Any foregoing method wherein the cancer is selected from adrenal cancer, bladder cancer, breast cancer, brain cancer, cervical cancer, colorectal cancer, endometrial cancer, kidney cancer, lip and oral cancer, liver cancer, lung cancer, melanoma, mesothelioma, non-small cell lung cancer, nonmelanoma skin cancer, oral cancer, ovarian cancer, pancreatic cancer, prostate cancer, sarcoma, skin cancer, small cell lung cancer, stomach cancer, and thyroid cancer.

1.19 Any foregoing method wherein the cancer is selected from duodenal adrenocarcinoma, cholangiocarcinoma, gastric cancer, liver cancer, ependymoma, medulloblastoma, pancreatic cancer, glioma, and choroid plexus tumor.

1.20 Any of methods 1-1.12 wherein the cancer is a blood cancer, e.g., selected from leukemia, lymphoma, and myeloma; for example, wherein the cancer is acute myeloid leukemia (AML).

1.21 Any foregoing method wherein the Compound of Formula (I) binds to a FGFR1OP2-FGFR1fusion protein, e.g., at amino acids corresponding to Leu48 of FGFR1OP2 and corresponding to Gly487 and Asp641 Mg²⁺MG776 of FGFR1.

1.22 Any foregoing method wherein the Compound of Formula (I) binds to a FGFR1OP2-FGFR1 binding complex, e.g., at amino acids corresponding to Arg62 and Gln70 of FGFR1OP2 and corresponding to Gln426 and Leu417 of FGFR1.

1.23 Any foregoing method wherein the dosage of the Compound of Formula (I) is an oral daily dose of 20 to 125 mg (e.g., 100mg BID).

1.24 Any foregoing method wherein the patient additionally receives radiation therapy and/or chemotherapy, e.g., before, during, or after treatment with a Compound of Formula (I).

1.25 Any foregoing method wherein the patient additionally receives radiation therapy, chemotherapy, immune checkpoint inhibitor (ICI) therapy or the combination thereof, e.g., before, during, or after treatment with a Compound of Formula (I).

1.26 Any foregoing method wherein the patient receives a kinase inhibitor in addition to the Compound of Formula (I), e.g., wherein the patient receives ponatinib.

The disclosure further provides a Compound of Formula (I), as hereinbefore described, in free or pharmaceutically acceptable salt form, for treatment of a cancer which (i) exhibits elevated levels of FGFR1OP2 and/or FGFR1; and/or (ii) is characterized by a translocation mutation expressing a FGFR1OP2 -FGFR1 fusion protein, e.g., for use in any of Methods 1, et seq., above.

The disclosure further provides the use of a Compound of Formula (I), as hereinbefore described, in free or pharmaceutically acceptable salt form, in the manufacture of a medicament for treatment of a cancer which (i) exhibits elevated levels of FGFR1OP2 and/or FGFR1; and/or (ii) is characterized by a translocation mutation expressing a FGFR1OP2-FGFR1 fusion protein, e.g., for use in any of Methods 1, et seq., above.

In a second embodiment, the disclosure provides a method (Method 2) of treating a cancer in a patient in need thereof, comprising administering to the patient

(i) an effective amount of a Compound of Formula (I), in free or pharmaceutically acceptable salt form:

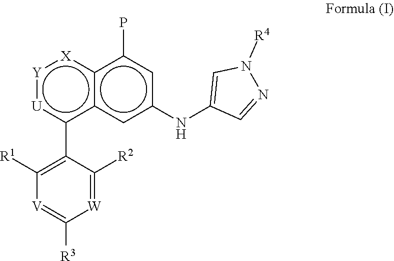

wherein, P is selected from a hydrogen or a deuterium;

X is selected from CH or S;

Y is selected from N or CR⁵;

U is selected from a chemical bond or CH;

V is selected from N or CH;

W is selected from N or CR⁶;

each of R¹, R², R³ and R⁶ is independently selected from the group consisting of a hydrogen, a deuterium, a halogen, a substituted or unsubstituted alkyl,

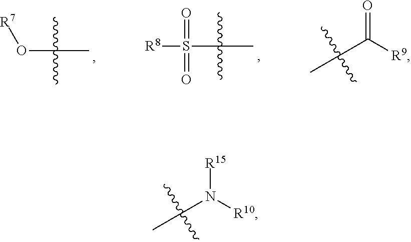

a cycloalkyl and a heterocycloalkyl; each of R⁷, R⁸, R⁹, R¹⁰ and R¹⁵ is independently selected from the group consisting of a hydrogen, a deuterium, a halogen, a hydroxyl, an amino, a substituted or unsubstituted alkyl, an alkoxy,

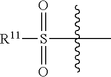

and a heterocycloalkyl; R¹¹ is a hydrogen, a deuterium or an alkyl; or R⁶, R² and the two atoms on the ring to which they are attached form a “substituted or unsubstituted 5- to 7-membered carbon heterocycle”; or, R⁶, R³ and the two atoms on the ring to which they are attached form a “substituted or unsubstituted 5- to 7-membered carbon heterocycle”; the heteroatom in “substituted or unsubstituted 5- to 7-membered carbon heterocycle” is selected from the group consisting of nitrogen, oxygen and sulfur;

R⁴ is a hydrogen, a deuterium, a substituted or unsubstituted alkyl, an alkoxy, a cycloalkyl, or a substituted or unsubstituted heterocycloalkyl;

R⁵ is a hydrogen, a deuterium, a halogen, or an alkyl;

in the definitions of R¹, R², R³ and R⁶, the “substituted” in “a substituted or unsubstituted alkyl” means to be substituted with the substituents selected from the group consisting of a halogen, a hydroxyl, an amino, an alkyl, an alkoxy,

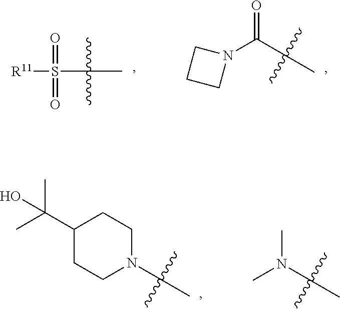

and a heterocycloalkyl, in the case when multiple substituents are present, the substituents are the same or different; R¹² is a hydrogen, a deuterium, or an alkyl;

in the definitions of R⁷, R⁸, R⁹, R¹⁰ and R¹⁵, the “substituted” in “a substituted or unsubstituted alkyl” means to be substituted with the substituents selected from the group consisting of a deuterium, a halogen, a hydroxyl. an amino, an alkyl. an alkoxy,

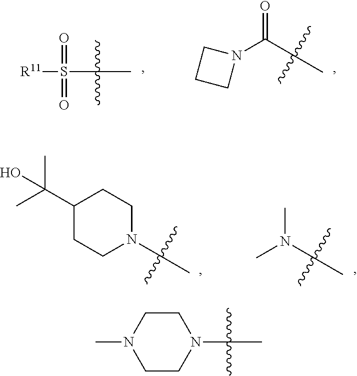

and a heterocycloalkyl, in the case when multiple substituents are present, the substituents are the same or different; R¹³ is a hydrogen or an alkyl;

in the definition of R⁴, the “substituted” in “a substituted or unsubstituted alkyl” and “a substituted or unsubstituted heterocycloalkyl” means to be substituted with the substituents selected from the group consisting of a hydroxyl, an alkyl,

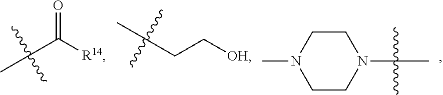

and heterocycloalkyl, in the case when multiple substituents are present, the substituents are the same or different; R¹⁴ is a hydrogen, an alkyl, a hydroxymethyl or an alkoxy;

the “substituted” in “substituted or unsubstituted 5- to 7-membered carbon heterocycle” means to be substituted with one or more than one alkyl

[e.g., as described in US10494378B2, the contents of which are incorporated herein by reference] in free or pharmaceutically acceptable salt form; and

(ii) an effective amount of an immune checkpoint inhibitor, e.g. an effective amount of an inhibitor of PD-1 or PD-L1.

For example, the disclosure provides

2.1Method 2 wherein the Compound of Formula (I) is Compound 1 in free or pharmaceutically acceptable salt form:

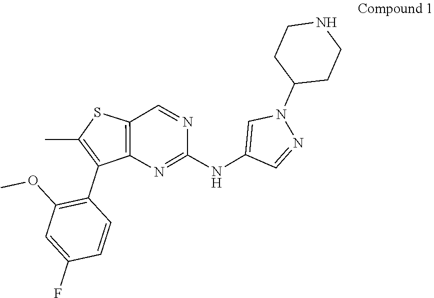 [SMILES: COc1cc(F)ccc1-c1c(C)sc2cnc(Nc3cnn(C4CCNCC4)c3)nc12]

2.2Method 2.1 wherein the Compound 1 is in pharmaceutically acceptable acid addition salt form.

2.3Method 2.2 wherein the pharmaceutically acceptable acid addition salt form of Compound 1 is selected from the fumarate, phosphate, tartrate, and adipate salts.

2.4Method 2.3 wherein the pharmaceutically acceptable acid addition salt form of Compound 1 is in crystalline form [e.g., as disclosed in WO2019228171A1, the contents of which are incorporated herein by reference].

2.5Method 2.3 or 2.4 wherein the acceptable acid addition salt form of Compound 1 is the hemifumarate.

2.6Any foregoing method wherein the cancer (i) exhibits elevated levels of FGFR1OP2 and/or FGFR1; and/or (ii) is characterized by a translocation mutation expressing a FGFR1OP2-FGFR1 fusion protein,

2.7Any foregoing method wherein the cancer exhibits elevated levels of FGFR1OP2 as measured using gene expression profiling, e.g., using RNA sequencing or real-time quantitative PCR (RT-qPCR).

2.8Any foregoing method wherein the cancer exhibits elevated levels of FGFR1OP2 as measured using an immunoassay for FGFR1OP2, e.g., using Western blotting, ELISA or in situ hybridization.

2.9Any foregoing method wherein the cancer exhibits elevated levels of FGFR1OP2 as measured using gene expression profiling, and the gene expression of FGFR1OP2 in the cancer is greater than five mRNA transcripts per million mRNA transcripts (TPM), e.g., at least 9 TPM, e.g., at least 10 TPM, e.g., at least 12 TPM., e.g., wherein TPM is calculated as described in Wagner G P, et al., “Measurement of mRNA abundance using RNA-seq data: RPKM measure is inconsistent among samples.” Theory Biosci. 2012 December; 131(4):281-5 and Abrams Z B, Johnson T S, Huang K, Payne P R O, Coombes K. “A protocol to evaluate RNA sequencing normalization methods. BMC Bioinformatics”. 2019; 20(Suppl 24):679. Published 2019 Dec. 20. doi:10.1186/s12859-019-3247-x.

2.10 Any foregoing method wherein the cancer expresses a FGFR1OP2- FGFR1 fusion protein; e.g. wherein the FGFR1OP2-FGFR1 fusion protein exhibits constitutive kinase activity, dimerization induction, constitutive signal transduction, and/or transforming activity; e.g., a protein wherein the first 2 coiled-coil domains of FGFR1OP2 are fused to the carboxy terminal part of FGFR1, including its tyrosine kinase domain, e.g., comprising 132 amino acids from FGFR1OP2 and 394 amino acids from FGFR1.

2.11 Any foregoing method wherein the cancer expresses a FGFR1OP2-FGFR1 fusion protein which exhibits constitutive kinase activity, e.g., wherein the fusion results from a chromosomal translocation to form a gene encoding the FGFR1OP2-FGFRlfusion protein.

2.12 Any foregoing method wherein the cancer expresses a FGFR1OP2- FGFR1 fusion protein as detected using an immunoassay for FGFR1OP2- FGFR1 fusion protein, e.g., using Western blotting, in situ hybridization, or ELISA.

2.13 Any foregoing method wherein the cancer expresses a FGFR1OP2- FGFR1 fusion protein as detected using gene expression profiling, e.g., using RNA sequencing or real-time quantitative PCR (RT-qPCR).

2.14 Any foregoing method wherein the cancer expresses a FGFR1OP2- FGFR1 fusion protein as detected using PCR or DNA probes to detect a mutation resulting in a gene encoding a FGFR1OP2-FGFR1 fusion protein.

2.15 Any foregoing method comprising the steps of

obtaining a biological sample, selected from blood or tumor tissue, from the patient, wherein the biological sample is believed to contain cancer cells;

either (i) detecting elevated levels of FGFR1OP2 expression in the biological sample using gene expression profiling and/or an immunoassay; or (ii) detecting FGFR1OP2- FGFR1 fusion protein or a gene encoding FGFR1OP2- FGFR1 fusion protein in the biological sample using gene expression profiling, PCR, DNA probe, or immunoassay; and

administering an effective dose of a Compound of Formula (I), e.g., of Compound 1, in free or pharmaceutically acceptable salt form to the patient if the biological sample exhibits either elevated levels of FGFR1OP2 expression or the presence of FGFR1OP2- FGFR1 fusion protein or a gene encoding FGFR1OP2- FGFR1 fusion protein.

2.16 Any foregoing method wherein the cancer exhibits FGFR1OP2 binding FGFR1 to exhibit constitutive kinase activity

2.17 Any foregoing method wherein the cancer is selected from carcinoma, sarcoma, melanoma, lymphoma, leukemia and myeloma (e.g., leukemia).

2.18 Any foregoing method wherein the cancer is a solid tumor.

2.19 Any foregoing method wherein the cancer is selected from adrenal cancer, bladder cancer, breast cancer, brain cancer, cervical cancer, colorectal cancer, endometrial cancer, kidney cancer, lip and oral cancer, liver cancer, lung cancer, melanoma, mesothelioma, non-small cell lung cancer, nonmelanoma skin cancer, oral cancer, ovarian cancer, pancreatic cancer, prostate cancer, sarcoma, skin cancer, small cell lung cancer, stomach cancer, and thyroid cancer.

2.20 Any foregoing method wherein the cancer is selected from duodenal adrenocarcinoma, cholangiocarcinoma, gastric cancer, liver cancer, ependymoma, medulloblastoma, pancreatic cancer, glioma, and choroid plexus tumor.

2.21 Any of methods 1 — 1.12 wherein the cancer is a blood cancer, e.g., selected from leukemia, lymphoma, and myeloma; for example, wherein the cancer is acute myeloid leukemia (AML).

2.22 Any foregoing method wherein the Compound of Formula (I) binds to a FGFR1OP2-FGFR1fusion protein, e.g., at amino acids corresponding to Leu48 of FGFR1OP2 and corresponding to Gly487 and Asp641 MeMG776 of FGFR1.

2.23 Any foregoing method wherein the Compound of Formula (I) binds to a FGFR1OP2-FGFR1 binding complex, e.g., at amino acids corresponding to Arg62 and Gln70 of FGFR1OP2 and corresponding to Gln426 and Leu417 of FGFR1.

2.24 Any foregoing method wherein the dosage of the Compound of Formula (I) is an oral daily dose of 20 to 125 mg (e.g., 100mg BID).

2.25 Any foregoing method wherein the patient additionally receives radiation therapy and/or chemotherapy, e.g., before, during, or after treatment with a Compound of Formula (I).

2.26 Any foregoing method wherein the patient additionally receives radiation therapy, chemotherapy, immune checkpoint inhibitor (ICI) therapy or the combination thereof, e.g., before, during, or after treatment with a Compound of Formula (I).

2.27 Any foregoing method wherein the patient receives a kinase inhibitor in addition to the Compound of Formula (I), e.g., wherein the patient receives ponatinib.

2.28 Any foregoing method wherein the Compound of Formula (I) and the inhibitor of PD-1 or PD-L1 are administered within one week of one another.

2.29 Any foregoing method wherein the immune checkpoint inhibitor is selected from an antibody to PD-1, an antibody to PD-L1, a small molecule inhibitor targeting the interaction of PD-1 and PD-L1, and combinations thereof.

2.30 Any foregoing method wherein the immune checkpoint inhibitor is an antibody to PD-1 or PD-L1.

2.31 Any foregoing method wherein the immune checkpoint inhibitor is an anti-PD-1 antibody, e.g. selected from pembrolizumab (Keytruda®), nivolumab (Opdivo®), cemiplimab (Libtayo®), toripalimab, or sintilimab.

2.32 Any foregoing method wherein the immune checkpoint inhibitor is an anti-PD-L1 antibody, e.g., selected from atezolizumab, durvalumab, avelumab, and cemiplimab.

2.33 Any foregoing method wherein the immune checkpoint inhibitor is a small molecule inhibitor targeting the interaction of PD-1 and PD-L1.

2.34 Any foregoing method wherein the immune checkpoint inhibitor is a small molecule inhibitor selected from the inhibitors identified in WO2018006795 (US20190308957A1), WO2019128918, WO2020192570A1, CN202010939415.0 and CN202011414403.2, the contents of which applications are incorporated herein by reference in their entirety.

2.35 Any foregoing method wherein the immune checkpoint inhibitor is a combination of (i) a small molecule inhibitor targeting the interaction of PD-1 and PD-L1 and (ii) an antibody to PD-1 or PD-L1.

2.36 Any foregoing method wherein at least one immune checkpoint inhibitor is

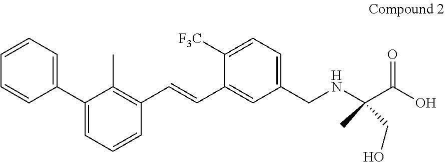 [SMILES: Cc1c(/C=C/c2cc(CN[C@@](C)(CO)C(=O)O)ccc2C(F)(F)F)cccc1-c1ccccc1]

in free or pharmaceutically acceptable salt form.

The disclosure further provides a Compound of Formula (I), as hereinbefore described, e.g., Compound 1, in free or pharmaceutically acceptable salt form, for use in combination with an effective amount of an immune checkpoint inhibitor, for treating cancer, e.g., for use in any of Methods 2, et seq., above, e.g., for use to enhance the effectiveness of the immune checkpoint inhibitor.

The disclosure further provides the use of a Compound of Formula (I), as hereinbefore described, e.g., Compound 1, in free or pharmaceutically acceptable salt form, in the manufacture of a medicament for use in combination with an effective amount of an immune checkpoint inhibitor, for treating cancer, e.g., for use in any of Methods 2, et seq., above.

The disclosure further provides immune checkpoint inhibitor, as hereinbefore described, in free or pharmaceutically acceptable salt form, for use in combination with an effective amount a Compound of Formula (I), as hereinbefore described, e.g., Compound 1, in free or pharmaceutically acceptable salt form, for treating cancer, e.g., for use in any of Methods 2, et seq., above.

The disclosure further provides the use of an immune checkpoint inhibitor, as hereinbefore described, in free or pharmaceutically acceptable salt form, in combination with an effective amount a Compound of Formula (I), as hereinbefore described, e.g., Compound 1, in free or pharmaceutically acceptable salt form, for treating cancer, e.g., for use in any of Methods 2, et seq., above.

The disclosure further provides a method of enhancing the effectiveness of an immune checkpoint inhibitor, as hereinbefore described, in a patient receiving immune checkpoint inhibitor therapy, comprising administering to the patient an effective amount of a Compound of Formula (I), as hereinbefore described, in free or pharmaceutically acceptable salt form.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

### BRIEF DESCRIPTION OF THE DRAWINGS

FIG. 1 is the RLU (ATP Enzyme activity) of cells from mini-PDX vehicles. The cancer cells derived from patients were embedded in the mini-PDX vehicles and implanted in the mice for cultivating 7 days. The mice were treated with MAX-40279(12mpk, PO, BID) or not. After 7 days, cells in mini-PDX vehicles were collected and ATP Enzyme activity was used to represent the cell viability.

FIG. 2 is the gene expression of candidate targets of MAX40279 in the patient. TPM were used to quantifying gene expression in the RNA-seq data.

FIG. 3 is the expression of FGFR/FLT3 related genes in the blood of patient before or after MAX40279 treatment. Gene expression levels were estimated using FPKM values. CODO means before treatment after in enrolled. C1D15 respects MAX40279 treatment for 15 days while C1D28 respects for 28 days.

FIG. 4 is the ratio of high-expressed FGFR1OP2 in all cancer. TheTPM>5 was defined as high expression. The ratio of high-expressed FGFR1OP2 in each cancer were calculated using TCGA data.

### DETAILED DESCRIPTION OF THE PREFERRED EMBODIMENT

The invention is further illustrated in the following examples, which are meant to be exemplary and not limiting.

### EXAMPLE 1

### Mini-PDX Assay and FGFR1OP2 Expression Levels in Solid Tumors

A mini patient-derived xenograft (mini-PDX) is used to assess the sensitivity of different tumors to treatment with Compound 1 (MAX-40279). In this experiment, clinical tumor samples from patients are treated in the mini-PDX by oral administration of Compound 1, and FGFR1OP2 expression level in those samples is analyzed.

Tumor tissue acquisition is approved by the ethics committees of each participating hospital and agreed to by each patient via written informed consent and is carried out according to state and institutional regulations on experimental use of human tissues. The patient samples are corrected if tumors >500 mm³ in size with a necrotic area <30% are used. Tumor tissues are then washed with Hank's balanced salt solution (HBSS) to remove non-tumor tissues and necrotic tumor tissue in a biosafety cabinet.

After the tumor tissues are morselized, they are digested with collagenase at 37 ° C. for 1-4 h. Cells are pelleted by centrifugation at 600g for 5 min followed by removal of blood cells and fibroblasts with magnetic beads. Cells are then washed with HBSS and filled into hollow fiber capsules (Shanghai LIDE Biotech Co., LTD). These capsules allow for the free entry and exit of small molecule drugs, large molecule antibody drugs and various growth factors less than 500KD, while tumor cells remain in the device. Capsules are implanted subcutaneously via a small skin incision with 3 capsules per mouse (5-week-old nu/nu mouse).

Mice bearing MiniPDX capsules are treated with Compound 1 for 7 days, once daily administered by oral administration at a dosage of 12 mg per kg.

Thereafter, the implanted capsules are removed, and tumor cell proliferation is evaluated using the CellTiter Glo Luminescent Cell Viability Assay kit (G7571, Promega, Madison, Wis., US) as instructed by the manufacturer. Luminescence is measured in terms of relative luminance unit (RLU) using a spectrophotometer (SpectraMax M3, Molecular Devices, Sunnyvale, Calif., US). Tumor cell growth inhibition (TCGI) (%) is calculated using the formula: TCGI %=(1−[mean RLU of the treatment group on day 7-mean RLU on day 0]/[Mean RLU of vehicle group on day 7−Mean RLU on day 0])×100%, Each experiment is done in sextuplicate and mean values are reported. The two-way ANOVA is adopted to stat p value.

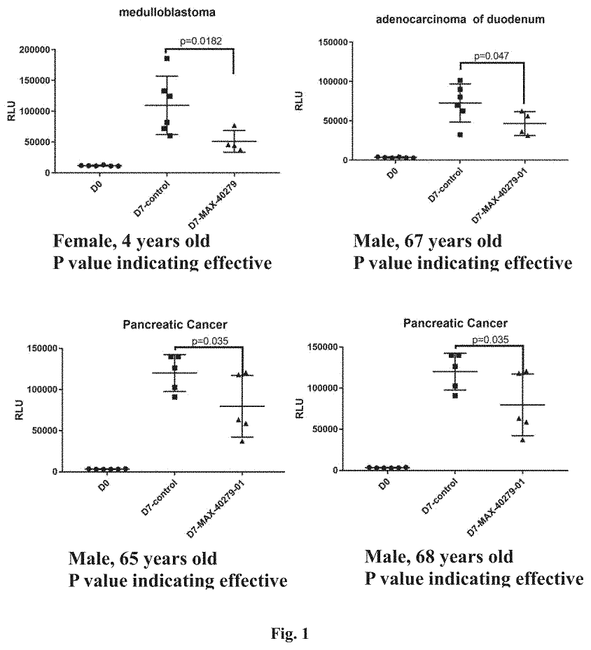

FIG. 1 depicts an initial screen of 4 samples treated in the mini-PDX, showing that Compound 1 provides effective TCGI after 7 days oral administration, compared to placebo.

Another two capsules from the treatment or vehicle group on day 7 are corrected to detect RNA and whole exon sequencing (WES).

The RNA extraction is carried out according to manufacturers' instructions via Single Cell Full Length mRNA-Amplification Kit (Vazyme biotech co., ltd.). Briefly, the single-cell lysate is thawed at 4° C. and centrifuged. Next, 0.5 μL of genomic DNA digestion mix (0.1 U of DNase I (Amplification Grade), and 2× DNase I Reaction Buffer in RNase-free water) is added to 1 μL of the single-cell lysate in a 96-well PCR plate and incubated at 25 ° C. for 5 min. After genomic DNA digestion, 0.5 μL of denaturing mix (8 mM EDTA and 0.02% NP40 in RNase-free water) is added to the digested samples, followed by incubation at 7° C. for 5 min to inactivate DNase I and desaturate the RNAs. The sample plate is immediately placed on ice. One microliter of the RT mix (3× VILO Reaction Mix and 3× SuperScript Enzyme Mix in RNase-free water) is added to the sample plate and incubated at 25° C. for 10 min, 42° C. for 60 min, and 85° C. for 5 min

The concentration of cDNA products is measured with a Qubit 2.0 Fluorometer (Life technologies, Carlsbad, USA). ing cDNA synthesized at last step is utilized to build sequencing library. Nextera® XT DNA Library Preparation Kit (Illumina #FC-131-1024) are used to library preparation.

The whole genome amplification (WGA) method is adopted to prepare WES library via Discover-sc® Single Cell WGA Kit (Vazyme biotech co., ltd.). All experimental operations conformed to the manufacturers' instructions strictly. The concentration of WGA products is measured with a Qubit 2.0 Fluorometer. Our experimental operations followed the manufacturers' protocols strictly and only reduced the input of DNA. A TruSeq DNA PCR-Free Library Preparation Kit (Cat. FC-121-3003, Illumina, San Diego, Calif., US) is applied for PCR-free library preparation. Libraries are prepared starting with 500 ng of total amplified DNA for each sample.

The size and quantity of the libraries are checked using a 2100 Bioanalyzer (Agilent Technologies). The sequencing is performed on Hiseq X ten platform via Paired-end 150 strategy, with approximately 30-40 million paired reads for the RNA sequencing (mRNA). Low quality reads (Phred quality score <20) and the first 20 bp of each read are trimmed Subsequently, the reads are aligned to the reference genome (GRCh37, UCSC release hg19) using the Burrow-Wheeler-Aligner (BWA) v0.7.7a algorithm. Then DESeq2 (version 3.10) is adopted to quantify the expression levels of genes including FGFR1OP2. BWA is used for accurate SNP and indels (insertion/deletions) identification. Somatic mutations are called using Mutect2 with default settings. The mutations in FGFRs and FGFR1OP2 are focused.

A high degree of correlation between FGFR1OP2 expression and responsiveness of tumors to treatment in the Mini-PDX assay is seen in Tables 1-6:

TABLE 1
FGFR1OP2 TPM vs P for Brain Cancers

| | | | | FGFR1OP2 |
|---|---|---|---|---|
| Patient | | | Efficacy | expression |
| # | Type | P Value | (P ≤ 0.05) | in TPM |
| 1 | CNS cancer | 0.28 | Non-effective | 0.529 |
| 2 | LGG | 0.94 | Non-effective | 1.643 |
| 3 | Glioma | 0.99 | Non-effective | 3.690 |
| 4 | Medulloblastoma | 0.54 | Non-effective | 4.068 |
| 5 | Medulloblastoma | 0.02 | effective | 19.065 |
| 6 | LGG | 0.03 | effective | 22.687 |
| 7 | Choroid plexus tumor | 0.02 | effective | 23.250 |
| 8 | Glioma | 0.04 | effective | 15.319 |
| 9 | Ependymoma | 0.0001 | effective | 25.098 |

TABLE 2
FGFR1OP2 TPM vs P for Pancreatic Cancers

| | | | FGFR1OP2 |
|---|---|---|---|
| Patient | | Efficacy | expression |
| # | P Value | (P ≤ 0.05) | in TPM |
| 1 | 0.85 | Non-effective | 0.000 |
| 2 | 0.23 | Non-effective | 8.219 |
| 3 | 0.28 | Non-effective | 4.483 |
| 4 | 0.51 | Non-effective | 7.485 |
| 5 | 0.09 | Non-effective | 3.151 |
| 6 | 0.03 | effective | 8.219 |
| 7 | 0.03 | effective | 21.000 |

Note:
(Patient 6 is an outlier, as the drug is effective with TPM < 9; in all other cases the drug is effective with TPM > 9).

TABLE 3
FGFR1OP2 TPM vs P for Liver Cancers

| | | | FGFR1OP2 |
|---|---|---|---|
| Patient | | Efficacy | expression |
| # | P Value | (P ≤ 0.05) | in TPM |
| 1 | 0.1589 | Non-effective | 5.179281 |
| 2 | 0.0275 | effective | 11.66406 |
| 3 | 0.0023 | effective | 19.84548 |

TABLE 4
FGFR1OP2 TPM vs P for Cholangiocarcinoma

| | | | FGFR1OP2 |
|---|---|---|---|
| Patient | | Efficacy | expression |
| # | P Value | (P ≤ 0.05) | in TPM |
| 1 | 0.9972 | Non-effective | 1.872244 |
| 2 | 0.0117 | effective | 22.30583 |
| 3 | 0.0412 | effective | 9.900412 |

TABLE 5
FGFR1OP2 TPM vs P for Gastric Cancer

| | | | FGFR1OP2 |
|---|---|---|---|
| Patient | | Efficacy | expression |
| # | P Value | (P ≤ 0.05) | in TPM |
| 1 | 0.87 | Non-effective | 0.684 |
| 2 | 0.002 | effective | 10.343 |

TABLE 6
FGFR1OP2 TPM vs P for Other Cancers

| | | | | FGFR1OP2 |
|---|---|---|---|---|
| Patient | | | Efficacy | expression |
| # | Type | P Value | (P ≤ 0.05) | in TPM |
| 1 | Colon cancer | 0.99 | Non-effective | 0.190 |
| 2 | Gallbladder cancer | 0.37 | Non-effective | 2.500 |
| 3 | Germ-cell tumor | 0.89 | Non-effective | 3.998 |
| 4 | Rhabdomyosoid tumor | 0.45 | Non-effective | 5.164 |
| 5 | Rhabdomyosoid tumor | 0.95 | Non-effective | 8.764 |
| 6 | Duodenal | 0.04 | effective | 9.335 |
| | adenocarcinoma | | | |

These data show that Compounds of Formula (I), particularly Compound 1, are effective treating a wide variety of cancer types having high expression of FGFR1OP2, e.g., greater than 9 TPM. Of the 30 tumor samples from patients, Compound 1 is effective in 13/30=43%. In all but one case, Compound 1 was effective with tumors expressing FGFR1OP2 TPM >9, and even in that one case, the expression level was still relatively high (8.219 TPM). If the cases are divided into those cases where Compound 1 was effective vs. those where Compound 1 was not effective, it is evident that FGFR1OP2 expression is much higher in the first group:

TABLE 7
Summary of correlation between efficacy
and FGFR1OP2 expression

| N = 30 | p < 0.05 | p > 0.05 |
|---|---|---|
| Average TPM | 16.77 | 3.63 |
| Median TPM | 19.07 | 3.69 |

### EXAMPLE 2

### Detection of FGFR1OP2 in Clinical Samples

The detection of FGFR1OP2 in clinical samples can be carried out in a variety of ways.

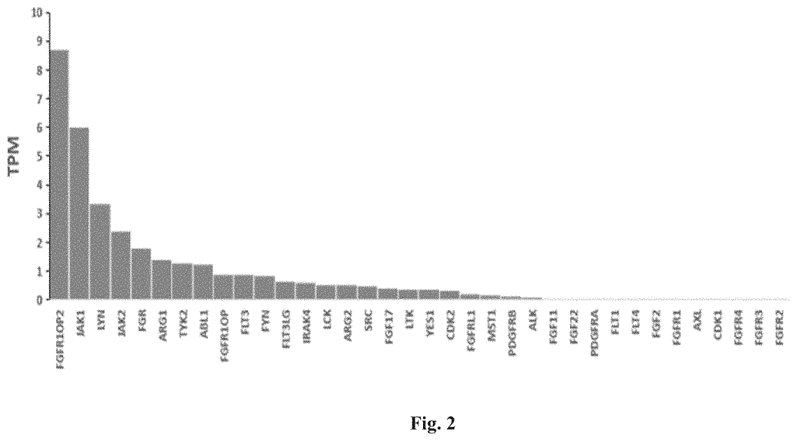

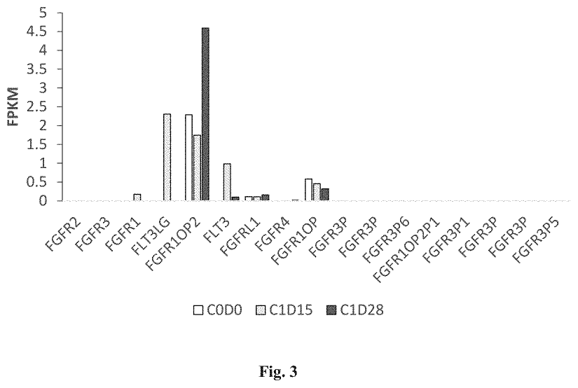

RNA Sequencing: Gene expression analysis by RNA sequencing (RNA-seq) is performed on RNA isolated from whole blood samples from AML patients, which is collected via PAXgene whole blood RNA tubes (BD) according to manufacturers' instructions. Then the total RNA is extracted using PAX Blood RNA Kit (BD). The next step is the creation of an RNA-Seq library using TruSeq RNA Library Prep Kit v2 (Illumina) and sequenced in Hiseq X ten platform. Expression values are calculated as TPM (Transcripts Per Million) and are used to determine differential expression of mRNAs in four time points (First PK run in (pre-dose), Cycle 1 Day 15 (pre-dose), Cycle 1 Day 28 (pre-dose), Treatment termination visit). A RNA-Seq analysis of a PRpatient expressing high levels of FGFR1OP2 is depicted in FIG. 2 (genomic expression in TPM prior to treatment) and FIG. 3 (genomic expression at days 0, 15 and 28, expression given as Fragments Per Kilobase of exon model per Million mapped fragments (FPKM)). (Complete Response, or CR, signifies that all target lesions have disappeared during the course of treatment, while Partial Response, or PR, signifies that decreases of at least 30% have been noted in the lesion that has the largest diameter, or LD.)

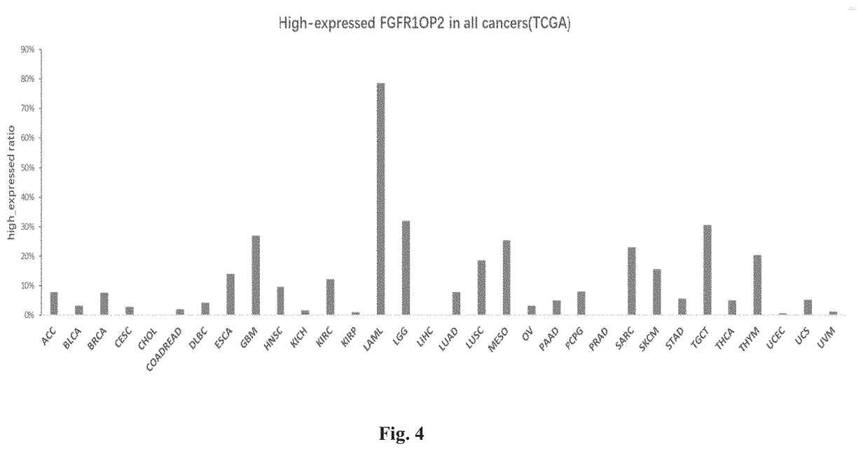

Elevated FGFR1OP2 expression is detected in more than 30 types of tumors. 80% of AML exhibits elevated FGFR1OP2. FIG. 4 provides data for some tumor types (key for tumor types given in Table 8):

TABLE 8

| | |
|---|---|
| ACC | Head & Neck Cancer |
| BLCA | Bladder Cancer |
| BRCA | Breast Cancer |
| CESC | Cervical Cancer |
| COADREAD | Colorectal Cancer |
| DLBC | B cell lymphoma |
| ESCA | Head & Neck Cancer |
| GBM | Brain Cancer |
| HNSC | Head & Neck Cancer |
| KIRC | Kidney Cancer |
| LAML | AML |
| LGG | Brain Cancer |
| LUAD | Lung Cancer |
| LUSC | Lung Cancer |
| MESO | Mesothelioma |
| OV | Ovarian Cancer |
| PAAD | Pancreatic Cancer |
| PCPG | Pheochromocytoma and Paraganglioma |
| SARC | Soft Tissue Sarcoma |
| SKCM | Skin Cancer |
| STAD | Stomach Cancer |
| TGCT | joint/tendon Cancer |
| THCA | Thyroid Cancer |
| THyM | Thymoma Cancer |
| UCS | uterine |

Other methods of detecting FGFR1OP2 in clinical samples include PCR assays and immunoassays, as follows.

RT-qPCR assay: The RNA is isolated from whole blood samples in PAXgene whole blood RNA tubes (BD) and extracted using PAX Blood RNA Kit (BD). A real-time quantitative PCR (RT-qPCR) analysis is carried out using the RV² Profiler PCR Array System (SA Biosciences Corp, Frederick, Md.) focusing on the blood clotting cascade and classical complement pathway according to the manufacturer's protocol. 500 ng of total RNA is used to produce cDNA using the RV First Strand Kit (Qiagen, Germantown, Md., USA), followed by qPCR assays using the RV² SYBR Green/Rox Mastermix Kit (Qiagen, Germantown, Md., USA) in an Applied Biosystems 7900HT (Applied Biosystems, Foster City, Calif., USA) under the recommended conditions. The RT-qPCR results are analyzed with SDS 2.3 software (Applied Biosystems, Foster City, Calif., USA). The primers of FGFR1OP2 are FGFR1OP2-F: AGCGAGTAGAAGCCATGAAACA and FGFR1OP2-R: CCCATAACTAACGTGGACCGT.

ELISA assay: The FGFR1OP2 ELISA kit (MBS9319814, MYBioSource) is adopted to analyze the protein expression in marrow fluids or serum in AML. Microtiter plates (96 well) are coated for 40 h at 4° C. with antigen (0.3 μg per well) diluted in phosphate-buffered saline (PBS) and subsequently blocked with 10% skim milk in PBS for 1 h. Patient sera are diluted 1:100 in PBS with 5% skim milk incubated for 1 h at room temperature, and washed three times with 0.1% Tween 20 in PBS and one time with PBS. Antigen at a 1,000-fold dilution is added. The same dilutions are used for rabbit anti-human IgG. After 1 h of incubation at room temperature, the plates are washed as previously described and the 2,2′-azinobis(3-ethylbenzthiazoline-6-sulfonate) (ABTS) substrate is added. After 30 min of incubation at room temperature, optical densities at 405 nm (OD405) are measured with a plate reader. Titers of antibody are expressed as the reciprocal of the highest dilution with a positive reaction.

In situ hybridization (ISH) assay: Tumor tissues are fixed in buffered 10% formalin and routinely stained with hematoxylin and eosin (H&E) and examined by a certified pathologist Immunofluorescence staining is performed according to previously described (Rothschild G, Zhao X, Iavarone A, Lasorella A. E Proteins and Id2 Converge on p57Kip2 To Regulate Cell Cycle in Neural Cells. Mol Cell Biol. 2006; 26:4351-4361.). The primary antibody is Abcam Anti-FGFR1OP2 antibody (ab229119, Abcam) and the dilution is 1:500. Confocal images acquired with a microscope are used to score positive cells. At least 500 cells are scored for each sample.

### EXAMPLE 3

### Clinical Efficacy of Compound 1

Data for seven cancer patients are summarized in Table 9. Stable Disease, or SD, signifies that there has been no significant decrease or increase in the size of target lesions (including tumors), based on the smallest sum lesion diameter (LD). Progressive Disease, or PD, signifies that there has been an increase of at least 20% in the sum of the LD of targeted lesions.

| | | Treatment | CT | FGFR1OP2 | FGFR1 wt |
|---|---|---|---|---|---|
| Dose | Patient Info | Days | Observation | TPM | (IHC) |
| 40 mg | Female, 69 y, | 85 | SD | 32 | 40% |
| | Vulvar squamous | | | | |
| | cell carcinoma | | | | |
| 70 mg | Female, 59 y, | 28 | PD | 7.5 | 50% |
| | CRC | | | | |
| | Female, 68 y, | 56 | PD | 27 | 30% |
| | Cholangiocarcinoma | | | | |
| | Female, 70 y, | 44 | PD | 24 | 25% |
| | Adenocarcinoma of | | | | |
| | gallbladder | | | | |
| 100 mg | Female, 67 y, | 41 | SD | 11 | 90% |
| | Squamous cell | | | | |
| | carcinoma | | | | |
| | of cervix | | | | |
| | Female, 69 y. | 28 | PD | 11 | 40% |
| | Ovarian cancer | | | | |
| | Female, 41 y, | On going | SD (20% | 5.3 | 80% |
| | CRC | (>112) | Reduction, 2 | | |
| | | | times of CT | | |
| | | | Examination) | | |

Patients are treated with Compound 1, given orally at the indicated daily dose. Treatment with Compound 1 stabilizes progression of the cancer in three of the seven patients. Although patients were not selected for high expression of FGFR1OP2 and FGFR1, the clinical response appears related to the expression levels of FGFR1OP2 and FGFR1. Squamous tumors seem particularly susceptible, as both patients with squamous tumors are SDs. Two of the three patients with SD are in 100 mg/day group, one of whom has 20% tumor reduction observed at the second and the fourth cycle by CT examination and is still under treatment. Adverse events are mild, so further dose escalation above 120 mg/day can be considered.

### EXAMPLE 4

### Combination with Anti-PD-1 Antibody

In a mouse model study, MAX-40279 is co-administered with anti-mPD-1 antibody in a 4T1 model. The 4T1 orthotopic breast cancer spontaneous metastasis mouse model is a transplanted tumor model, in which breast tumor cells are transplanted into the mammary fat pad to establish primary tumor nodules. The primary tumor can then be surgically removed as in human breast cancer patients. This particular model is known to be relatively resistent o treatment with therapies targeting PD-1/PDL-1.

The anti-mPD-1 monoclonal antibody is administered at a dose of 10 mg/kg by intraperitoneal (i.p.) injection, twice a week (b.i.w.). Compound 1 is administered at doses of 7, 10 and 15 mg/kg, orally, twice a day (b.i.d.). Based on clinical trial results for Compound 1, a dosage of 7 mg/kg b.i.d. in mice corresponds to 70 mg/day in humans; 10 mg/kg b.i.d. in mice corresponds to 100 mg/day in humans; 15 mg/kg b.i.d. in mice corresponds to 150 mg/day in humans. Results are provided as percent inhibition of tumor growth by volume relative to control (TGI).

| | | Avg. TGI, |
|---|---|---|
| | n | day 18 |
| Vehicle control | 10 | — |
| Anti-mPD-1, 10 mg/kg, ip, biw | 10 | 15.43 |
| Cmpd 1 7 mg/kg, po, bid + Anti-mPD-1, 10 mg/kg, | 10 | 34.27 |
| ip, biw | | |
| Cmpd 1 10 mg/kg, po, bid + Anti-mPD-1, 10 mg/kg, | 10 | 37.29 |
| ip, biw | | |
| Cmpd 1 15 mg/kg, po, bid + Anti-mPD-1, 10 mg/kg, | 10 | 40.82 |
| ip, biw | | |

This data supports that co-administration of Compound 1 enhances the treatment response to anti-PD-1/L1 antibody drugs, especially for the tumors with high level of FGFR1OP2 and/or FGFR1 expression.

1. A method of treating a cancer in a patient in need thereof, wherein the cancer (i) exhibits elevated levels of FGFR1OP2 and/or (ii) is characterized by a translocation mutation expressing a FGFR1OP2-FGFR1 fusion protein, comprising administering an effective amount of a Compound of Formula (I), in free or pharmaceutically acceptable salt form: 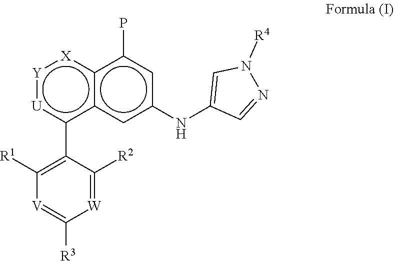
wherein, P is selected from a hydrogen or a deuterium;
X is selected from CH or S;
Y is selected from N or CR5;
U is selected from a chemical bond or CH;
V is selected from N or CH;
W is selected from N or CR6;
each of R1, R2, R3 and R6 is independently selected from the group consisting of a hydrogen, a deuterium, a halogen, a substituted or unsubstituted alkyl, 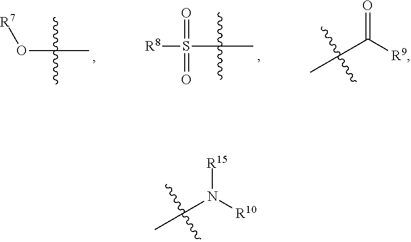
a cycloalkyl and a heterocycloalkyl; each of R7, R8, R9, R10 and R15 is independently selected from the group consisting of a hydrogen, a deuterium, a halogen, a hydroxyl, an amino, a substituted or unsubstituted alkyl, an alkoxy, 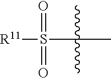
and a heterocycloalkyl; R11 is a hydrogen, a deuterium or an alkyl; or R6, R2 and the two atoms on the ring to which they are attached form a “substituted or unsubstituted 5- to 7-membered carbon heterocycle”; or, R6, R3 and the two atoms on the ring to which they are attached form a “substituted or unsubstituted 5- to 7-membered carbon heterocycle”; the heteroatom in “substituted or unsubstituted 5- to 7-membered carbon heterocycle” is selected from the group consisting of nitrogen, oxygen and sulfur;
R4 is a hydrogen, a deuterium, a substituted or unsubstituted alkyl, an alkoxy, a cycloalkyl, or a substituted or unsubstituted heterocycloalkyl;
R5 is a hydrogen, a deuterium, a halogen, or an alkyl;
in the definitions of R1, R2, R3 and R6, the “substituted” in “a substituted or unsubstituted alkyl” means to be substituted with the substituents selected from the group consisting of a halogen, a hydroxyl, an amino, an alkyl, an alkoxy 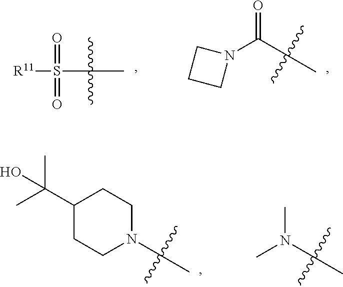
and a heterocycloalkyl, in the case when multiple substituents are present, the substituents are the same or different; R12 is a hydrogen, a deuterium, or an alkyl;
in the definitions of R7, R8, R9, R10 and R15, the “substituted” in “a substituted or unsubstituted alkyl” means to be substituted with the substituents selected from the group consisting of a deuterium, a halogen, a hydroxyl, an amino, an alkyl, an alkoxy, 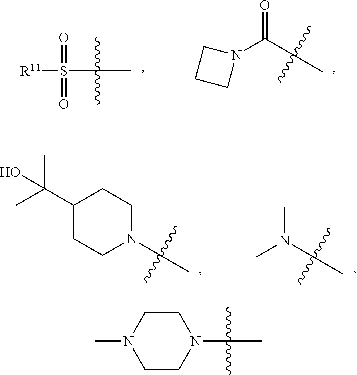
and a heterocycloalkyl, in the case when multiple substituents are present, the substituents are the same or different; R13 is a hydrogen or an alkyl;
in the definition of R4, the “substituted” in “a substituted or unsubstituted alkyl” and “a substituted or unsubstituted heterocycloalkyl” means to be substituted with the substituents selected from the group consisting of a hydroxyl, an alkyl, 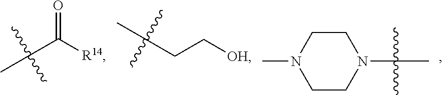
and heterocycloalkyl, in the case when multiple substituents are present, the substituents are the same or different; R14 is a hydrogen, an alkyl, a hydroxymethyl or an alkoxy;
the “substituted” in “substituted or unsubstituted 5- to 7-membered carbon heterocycle” means to be substituted with one or more than one alkyl.

2. The method of claim 1 wherein the Compound of Formula (I) is Compound 1, in free or pharmaceutically acceptable salt form:

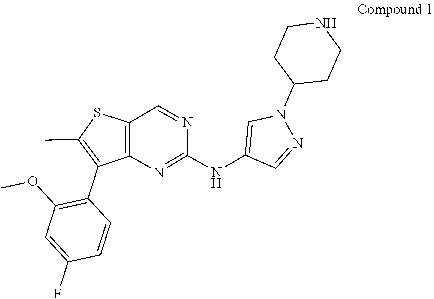 [SMILES: COc1cc(F)ccc1-c1c(C)sc2cnc(Nc3cnn(C4CCNCC4)c3)nc12]

3. The method of claim 2 wherein Compound 1 is in pharmaceutically acceptable acid addition salt form.

4. The method of claim 3 wherein the pharmaceutically acceptable acid addition salt form of Compound 1 is selected from the fumarate, phosphate, tartrate, and adipate salts.

5. The method of claim 3 wherein the pharmaceutically acceptable acid addition salt form of Compound 1 is in crystalline form.

6. The method of claim 1 wherein the cancer exhibits elevated levels of FGFR1OP2.

7. The method of claim 1 wherein the cancer exhibits elevated levels of FGFR1OP2 as measured using gene expression profiling, and the gene expression of FGFR1OP2 in the cancer is greater than five mRNA transcripts per million mRNA transcripts (TPM), e.g., at least 9 TPM, e.g., at least 10 TPM, e.g., at least 12 TPM.

8. The method of claim 1 wherein the cancer expresses a FGFR1OP2- FGFR1 fusion protein.

9. The method of claim 1 wherein the cancer is selected from carcinoma, sarcoma, melanoma, lymphoma, leukemia and myeloma, e.g., solid tumor, e.g., adrenal cancer, bladder cancer, breast cancer, brain cancer, cervical cancer, colorectal cancer, endometrial cancer, kidney cancer, lip and oral cancer, liver cancer, lung cancer, melanoma, mesothelioma, non-small cell lung cancer, nonmelanoma skin cancer, oral cancer, ovarian cancer, pancreatic cancer, prostate cancer, sarcoma, skin cancer, small cell lung cancer, stomach cancer, or thyroid cancer, e.g., duodenal adrenocarcinoma, cholangiocarcinoma, ependymoma, medulloblastoma, glioma, and choroid plexus tumor, or a blood cancer, e.g., selected from leukemia, lymphoma, and myeloma, e.g., acute myeloid leukemia (AML).

10. The method of claim 1 wherein the dosage of the Compound of Formula (I) is an oral daily dose of 20 to 125 mg.

11. The method of claim 1 wherein the patient additionally receives radiation therapy, chemotherapy, immune checkpoint inhibitor therapy or the combination thereof.

12. A method of treating a cancer in a patient in need thereof, wherein the cancer (i) exhibits elevated levels of FGFR1OP2 and/or (ii) is characterized by a translocation mutation expressing a FGFR1OP2-FGFR1 fusion protein, comprising the steps of
a) obtaining a biological sample, selected from blood or tumor tissue, from the patient, wherein the biological sample is believed to contain cancer cells;
b) either (i) detecting elevated levels of FGFR1OP2 expression in the biological sample using gene expression profiling and/or an immunoassay; or (ii) detecting FGFR1OP2- FGFR1 fusion protein or a gene encoding FGFR1OP2- FGFR1 fusion protein in the biological sample using gene expression profiling, PCR, DNA probe, or immunoassay; and
c) administering an effective dose of a Compound of Formula (I), as hereinbefore described, e.g., of Compound 1, in free or pharmaceutically acceptable salt form to the patient if the biological sample exhibits either elevated levels of FGFR1OP2 expression or the presence of FGFR1OP2- FGFR1 fusion protein or a gene encoding FGFR1OP2- FGFR1 fusion protein.

13. The method of claim 1 further comprising administering an effective amount of an immune checkpoint inhibitor.

14. (canceled)

15. A method of enhancing the effectiveness of an immune checkpoint inhibitory in a patient receiving immune checkpoint inhibitor therapy, comprising administering an effective amount of a Compound of Formula (I), in free or salt form, as defined in claim 1.

16. (canceled)

17. (canceled)

18. The method of claim 4 wherein the pharmaceutically acceptable acid addition salt form of Compound 1 is the hemifumarate.

19. The method of claim 10 wherein the dosage of the Compound of Formula (I) is 100mg BID.

20. The method of claim 13 wherein the immune checkpoint inhibitor is an inhibitor of PD-1 or PD-L1.

21. The method of claim 13 wherein the immune checkpoint inhibitor is antibodies to PD-1 or PD-L1, or small molecule inhibitors targeting the interaction of PD-1 and PD-L1.

22. The method of claim 15 wherein the immune checkpoint inhibitor is an inhibitor of PD-1 or PD-L1.

23. The method of claim 15 wherein the immune checkpoint inhibitor is antibodies to PD-1 or PD-L1, or small molecule inhibitors targeting the interaction of PD-1 and PD-L1.
